(19) United States
(12) Patent Application Publication
(10) Pub. No.: US 2022/0152412 A1
(43) Pub. Date: May 19, 2022

(54) SUBLINGUAL ULTRA VIOLET WAND DEVICE

(71) Applicant: Thomas Perez, Chicago, IL (US)

(72) Inventor: Thomas Perez, Chicago, IL (US)

(21) Appl. No.: 16/951,433

(22) Filed: Nov. 18, 2020

(51) Int. Cl. A61N 5/06

(52) U.S. Cl. CPC A61N 5/0603; A61N 5/0624; A61N 2005/0606; A61N 2005/0626; A61N 2005/0668; A61N 2005/0661; A61N 2005/0651

(57) ABSTRACT

A sublingual, ultra violet (UV) light device is disclosed. The light device is comprised of a cold cathode ray tube and a light emitting diode (LED) and a mouth piece. Embodiments of the device can include an onboard controller, a handle and can be powered by a multitude of sources. The light device combines light from a cold cathode ray tube with light from LEDs to enhance the various wavelength exposures to the user. An object of the invention is to enhance conventional cold cathode ray tube immunotherapy with complementary LED light.

### FIELD OF THE INVENTION

The invention relates to LED immunotherapy. More specifically, it relates to a device that combines LED and cathode ray tube light for enhanced immunotherapy treatment.

### BACKGROUND

Visible and red light therapy can deliver therapeutic wavelengths of natural light to the body's skin and cells. Such therapy, commonly referred to as light therapy and photo-biomodulation offers a wide range of health benefits that can include wound healing, pain relief and skin rejuvenation. Red light therapy exposure can stimulate adenosine triphosphate within the body to boost collagen production and repair damaged tissues. One of the major benefits of red light therapy is the effect on the energy center within the body's cells. This energy center is made up of mitochondria in the cells. Mitochondria produces the energy or fuel that our cells need to function and ultimately survive. As humans age, cells in the body become unwell, suffer from stress and can become slow to heal, inflamed or they simply die off. The body's cellular structure is the catalyst for most diseases and degenerative conditions. Red light therapy can increase nitric oxide, a compound that heals the cells and increases the blood flow which may help to stimulate healing and strengthen your immune system to defend against disease; regulate blood pressure and reduce inflammation. Nitric oxide is also crucial to the health of arteries. Red light therapy can help the mitochondria to produce the fuel or energy it requires for healthy cells and enhanced cell production. LED light can penetrate beyond the top layers of the skin to between eight and 10 millimeters which can influence the nervous system, metabolic processes as well as the immune system And because unhealthy cells are the cause of most diseases and degenerative conditions, it stands to reason that healthy cells can lead to better health, reduced stress levels and feelings of wellbeing.

Ultraviolet light is applied either to the patient's skin or directly to the blood. If the UV light is applied to the skin it is typically provided to the patient's skin either with a wrap or lamp. UV light is commonly used to treat jaundiced babies. Because infant's skin is thin and the blood vessels are close to the surface, UV light is typical applied to the skin when treating jaundiced babies. Applying the UV light directly to a patient's blood supply is known as photoluminescence or UV blood illumination (UBI). UV blood illumination increases oxygen, destroys toxins and boosts the immune. In prior art UBI, a small amount of blood is drawn from the patient, up to about 250 cc. The body has about 5.6 L of blood. The blood that is drawn travels through a cuvette or glass chamber. The blood is repeatedly illuminated with UV light and then returned to the body. The process is repeated, typically a day or several days later. These treatments are time consuming, and require regular trips to a medical facility. In addition, trained personal must be available to provide the treatments.

There is a need for a method of providing UV light to a patient's entire blood supply, not just a small portion of it. There is a need for a system that is convenient for the patient, which does not require regular doctor visits. There is a need for a simple system that can be used by the patient in his home. The body's ability to combat injury, inflammation and skin conditions may also be enhanced. New areas of research into the benefits of red-light therapy have been increasing however, prior art are not small enough to target specific areas of the body. Other prior art may disclosed a cathode ray bulb inserted into the mouth to deliver therapeutic light to the blood; however, they do not incorporate cathode ray tubes with LED lights for immunotherapy. There is a need for a system that allows for round the clock treatments or other regular treatments such as pulsed treatment or automatic periodic treatments.

There is a need for a blood illuminator that reduces the risk of infection from removing blood.

There is a need for a system that allows the blood to be treated on an as needed basis, such as based on how the patient is feeling at a particular time.

There is a need for a system that combines cathode ray tube light with LED light to enhance light treatment therapy sublingually.

### SUMMARY OF THE INVENTION

The present invention is a light device or a portable light wand that irradiates the mucous membrane under the tongue. The light device can have an onboard battery or other power supply. The light source emits light at one or more therapeutic wavelengths. Preferably, the light is UV light at one or more therapeutic wavelengths. The UV light source is typically a cold, cathode ray tube that emit UV-A or UV-C light or a combination of UV-A and UV-C light.

An object of the invention is to enhance phototherapy by combining light from a cold, cathode ray tube and an LED light. The light wand or device is inserted into the user's mouth, preferably under the tongue. Capillaries are plentiful and close to the surface under the tongue, thus illuminating the blood. A shell having a cold cathode fluorescent bulb with LED affixed thereon is inserted under a patient's tongue to irradiate the mucus membrane and provide the UV light to the blood.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention. As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The objects features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF THE FIGURES

FIG. 1 showing an embodiment of the device inserted into a user's mouth.

FIG. 2 showing a perspective view of the device.

FIG. 3 showing an exploded view of the device.

FIG. 4 showing a top view of the device.

FIG. 5 showing a side view of the device.

FIG. 6 showing a bottom view of the device.

FIG. 7 showing a front view of the device.

FIG. 8 showing a rear, perspective view of the device.

FIG. 9 showing a bottom, perspective view of the device.

FIG. 10 showing a front, perspective view of the device.

### DETAILED DESCRIPTION OF THE FIGURES

Light at one or more therapeutic wavelength, such as ultraviolet light, is used to treat many diseases including infections, poisoning, fatigue, allergies, hepatitis, cancer and HIV. UV light increases the oxygen combining power of the blood, destroys toxins, viruses, fungi, bacteria, and boosts the immune system. UV light also sterilizes the blood and acts as an antibiotic. Preferably, UV light at one or more therapeutic wavelength is utilized in the present invention. More preferably the light is either UV-A or UV-C light is utilized in the present invention. For some conditions and/or diseases UV-A light is more effective than UV-C and for other conditions and/or diseases UV-C light is more effective than UV-A light. The wavelengths or wavelengths of light to be used to treat the patient are selected based on the wavelength or wavelength that will best treat the condition or disease of the patient.

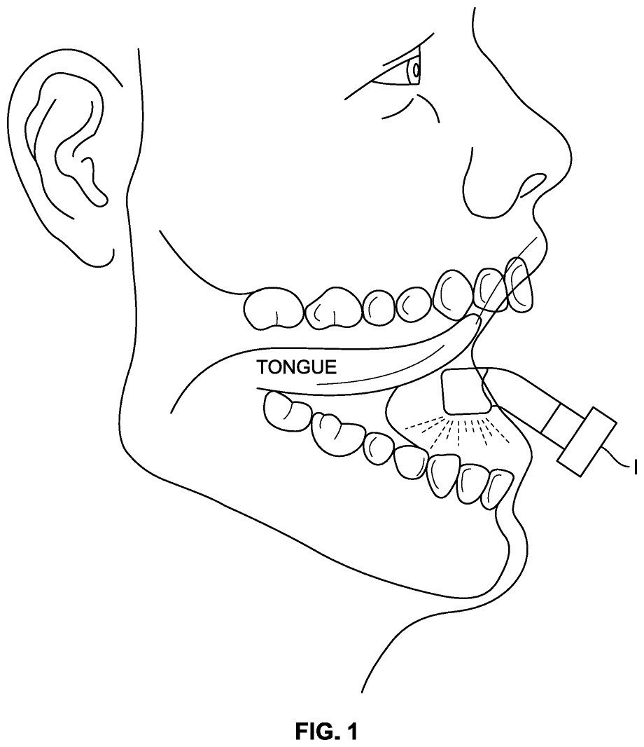

FIG. 1 shows a preferred embodiment of the invention. The invention is a light device 1 that is inserted into a patient's mouth to directly supply light and/or radiation at a therapeutic wavelength(s), preferably UV light, to the patient's blood supply. The light device 1 allows therapeutic wavelength(s) of light to be supplied directly to the blood by means of light exposing the patient's mucous membrane. Instead of treating only a maximum of 250 cc of blood, larger amounts of blood or even the entire blood supply can be treated. The 5.6 L of blood in a human body circulates through the body about 3 times every minute. Thus, large amounts of blood can be treated with photoluminescence. In other embodiments the Light device 1 could be turned on and off manually. Alternatively, the light device 1 could be automatically turn off the light source after a set treatment time, such as 20 minutes. Light device 1 could have a controller such as a computer or other smart interface that limits the number of treatments given time period, limits the total amount of treatment time in a given time period, automatically provides treatments, pulses the light source, or provides only particular wavelengths.

A computer or other smart interface could keep a treatment record. The computer or other smart interface could communicate wirelessly, via the Internet or through other electronic means to automatically update the doctor's treatment records. Computer preferably can automatically adjust treatment time, wavelength or other factors based on patient input, doctor orders or other data.

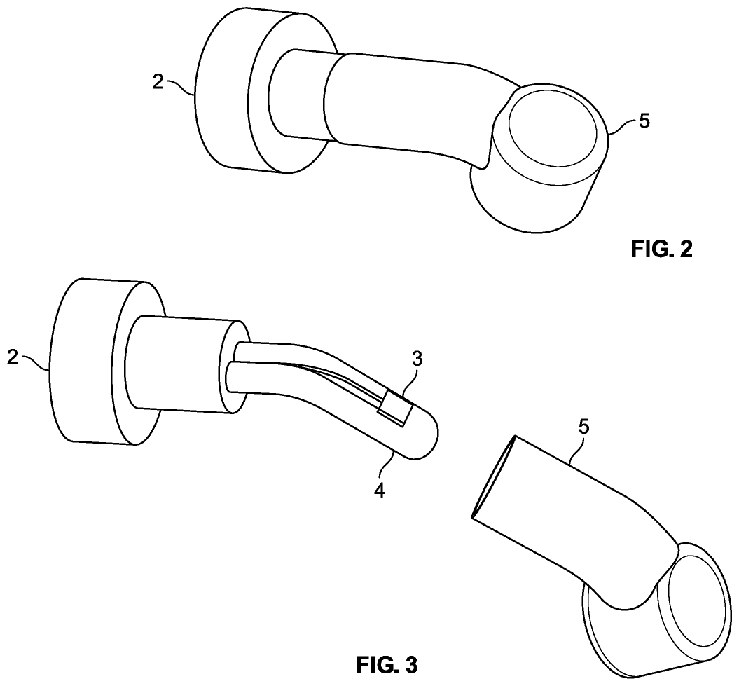

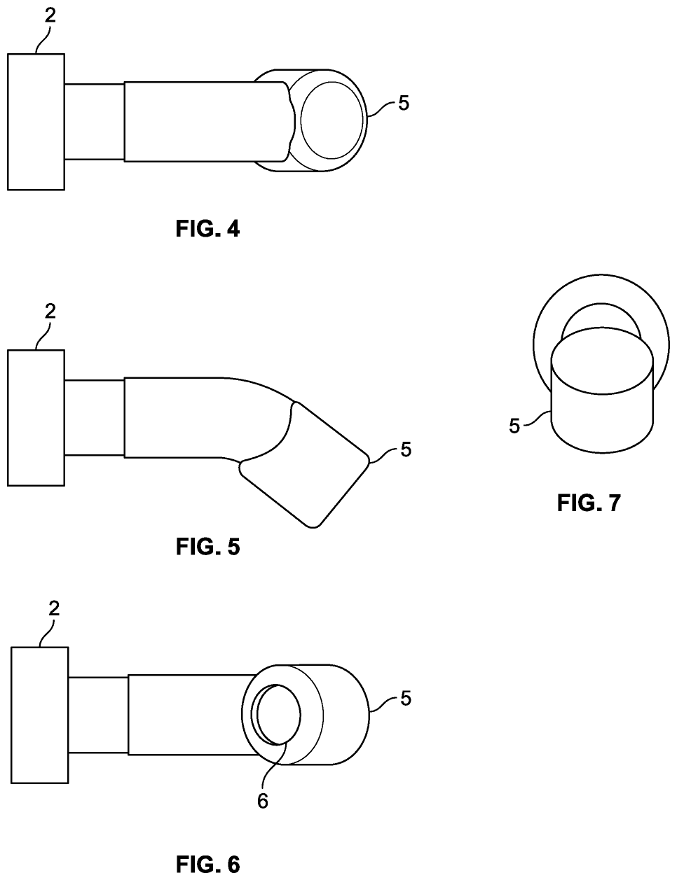

In the preferred embodiment of the invention, light at a therapeutic wavelength is administered under the tongue. The capillaries under the tongue are close to the surface. These capillaries are very sensitive. Capillary exposure of the mucus membrane is significantly greater than other exposed body surfaces. The greater capillary exposure allows for greater penetration of the ultraviolet spectrum. FIGS. 2 and 3 showing the light device comprised of a cold cathode ray tube 4 comprises a loop member and a shaft that is bent to an angle of at least, but not limited to, a 30-degree angle for holding and aligning the light source under the tongue. The cold cathode ray tube 4 also having at least one LED 3 disposed at the end and being affixed by a transparent strap thereon and being anchored in cylinder sleeve 2 (made of a rigid material such as but not limited metal, plastic and the like). Light device 1 comprises a cold cathode ray tube 4 or other bulb such as cold fusion bulb. The cold cathode ray tube 4 can be housed in a waterproof shell 5. The light device 1 be adapted to be placed directly under a patient's tongue. FIGS. 4-7 and 10 show various view of the device.

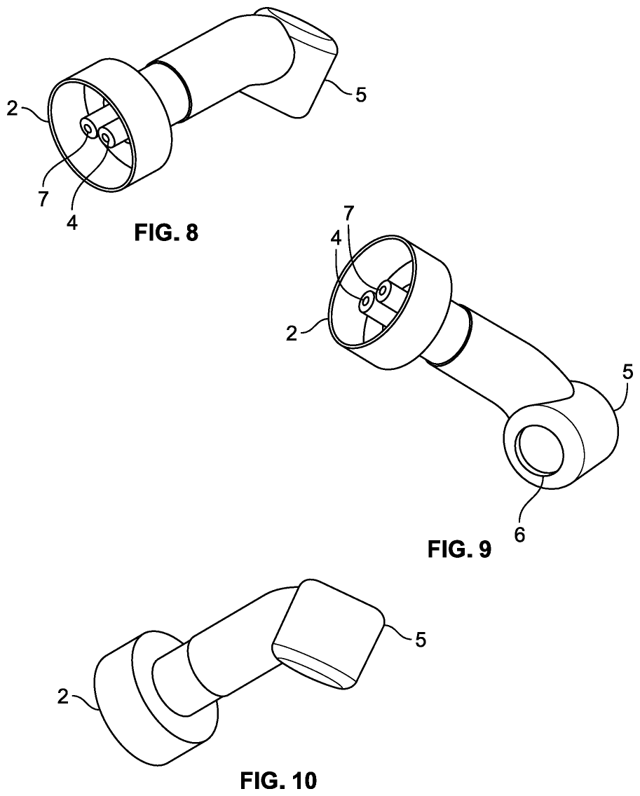

The waterproof shell 5 can be oriented in substantially parallel alignment with the back of the tongue or can be aligned substantially perpendicular to the back a of tongue. The waterproof shell 5 is placed under tongue so that the mucous membrane where the capillaries are close to the surface receives the irradiation. Preferably, the shell is made of a material which allows emission of UV light. Optionally, the waterproof shell 5 has a window the permits the emission of the UV radiation. If the waterproof shell 5 has a window 6, it is preferable that the window 6 be oriented toward the bottom of the mouth. Said window 6 being made of a transparent material such as but not limited to glass, crystal, Plexiglas and the like and allowing light to transfer to the mouth of a user. One or more low voltage cables 7 used to power the cold cathode ray tube 4 and LED 3. Low voltage cables 7 are preferably attached to the shell at a waterproof connection as shown in FIGS. 8 and 9. The low voltage cables 7 may have a shaft with one or more pins adapted to mate with the low voltage cable with a female connection end. Other known electrical connections can be utilized. It is preferable that cathode ray tube 4 and LED 3 may be removable for cleaning, sterilization and/or replacement of the bulb. In another alternative embodiment, the cathode ray tube 4, LED 3 and low voltage cables 4 are an integral unit and connected to a power supply. The cathode ray tube 4 and LED 3 having pins adapted to mate with the low voltage cable. In yet another alternative embodiment, the cathode ray tube 4, LED 3 and power supply are an integral unit. The light is preferably UV-A, UV-C or a combination thereof. The light cathode ray tube 4 preferably has a loop shape. The cathode ray tube 4 also preferably has an angle to make it more comfortable for the patient. Optionally, waterproof shell 5 is made of a flexible material such as but not limited to rubber, silicone and the like. This material will be more comfortable for the patient. Optionally, the flexible material allows the waterproof shell 5 to mold to the patient's mouth. The cathode ray tube 4 and LED 3 allows light at one or more therapeutic wavelengths to be supplied directly to the blood. Instead of treating only a maximum of 250 cc of blood, larger amounts of blood or even the entire blood supply can be treated. The 5.6 L of blood in a human body circulates through the body about 3 times every minute. Thus, large amounts of blood can be treated with photoluminescence.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention.

I claim:

1. A light device comprised of the following parts:
a.) a cold cathode ray tube adapted to emit radiation at one or more therapeutic wavelengths and said light source adapted to be placed under a patient's tongue;
b.) an LED adapted to emit radiation at one or more therapeutic wavelengths and said light source adapted to be placed under a patient's tongue;
c.) cylinder sleeve to anchor and support the cold cathode ray tube and LED;
d.) a waterproof shell for preventing saliva from reaching the cold cathode ray tube and LED, and
e.) a power supply to energize the cold cathode ray tube and LED.

2. The light device of claim 1 wherein the cold cathode ray tube UV radiation is UV-A radiation, UV-C radiation or a combination thereof.

3. The light device of claim 1 wherein the LED UV radiation is UV-A radiation, UV-C radiation or a combination thereof.

4. The light device of claim 1 wherein the cold cathode ray tube comprises a loop member and a shaft, said shaft electrically connected to the power supply transparent and a second end portion is connected to the power supply.

5. The light device of claim 1 wherein a low voltage cable connects the power supply and the cold cathode ray tube and LED.

6. The light device of claim 1 wherein the LED being affixed to a distal end of the cold cathode ray tube with strapping.

7. The light device of claim 1 wherein at least a portion of the cold cathode tube is transparent or translucent.

8. The light device of claim 1 wherein the cold cathode ray tube and LED having at least one pin adapted to mate with the low voltage cable.

9. The light device of claim 1 wherein the light source has a first portion and a second portion and said first portion is angled relative to said second portion.

10. The light device of claim 1 wherein the cold cathode ray tube is angled relative to an end portion of the shaft.

11. The light device of claim 1 wherein the wherein the waterproof shell is made of a flexible material.

12. The light device of claim 1 wherein the wherein the waterproof shell having a window on a distal.

13. A method of illuminating blood comprising:
a.) inserting the light device of claim 1 under a patient's tongue;
b.) directing radiation of one or more therapeutic wavelength to the patient's mucus membrane; and
c.) illuminating the mucus membrane for a period of time with light.

14. The method of illuminating blood of claim 13 further wherein the therapeutic wavelength is one or more UV wavelengths.
